(19) United States
(12) Patent Application Publication
(10) Pub. No.: US 2002/0082317 A1
(43) Pub. Date: Jun. 27, 2002

(54) Dental adhesive compositions with desensitizing agents

(75) Inventors: Carey Lyons, Englewood, NJ (US); Milton Hodosh, Providence, RI (US)

Correspondence Address: Michele J. Young, Salter & Michaelson, 321 south Main Street, Providence, RI, 02903, US

(73) Assignee: SULTAN CHEMISTS, INC.

(21) Appl. No.: 09/997,796

(22) Filed: Nov. 30, 2001

Related U.S. Application Data
(63) Continuation of application No. 09/394,775, filed on Sep. 13, 1999, now abandoned.
(60) Provisional application No. 60/100,013, filed on Sep. 11, 1998.

(51) Int. Cl. A61K 6/08

(52) U.S. Cl. 523/116

(57) ABSTRACT

Dental cements containing desensitizing agents, preferably potassium-containing desensitizing agents, are described. The cements favorably provide decreased post-application sensitivity upon application compared to prior art formulations. Methods for preparing these cements are also described, as are methods for decreasing the incidence and the severity of post-application sensitivity using the desensitizing cements of the invention.

### REFERENCE TO PRIOR APPLICATIONS

This application is a continuation application of copending U.S. patent application Ser. No. 09/394,775 filed Sep. 13, 1999 and claims priority thereof.

### FIELD OF THE INVENTION

The present invention relates to dental resin compositions (dental cements) containing a desensitizing agent. Methods of preparing dental adhesive compositions and methods of reducing the incidence or severity of tooth sensitization or post-application pain are also provided.

### BACKGROUND OF THE INVENTION

All references cited herein are incorporated by reference in their entireties.

There is a growing need in the dental art for biocompatible materials which do not produce undesirable post-application side effects, such as tooth sensitivity. Materials used in dentistry have shown significant improvements over the years, particularly dental adhesive compositions or dental cements.

In the past, zinc phosphate and zinc-eugenol cements were used, but the undesirable properties of such products are well documented in the literature. Polycarboxylate cements eventually became the primary class of cements based in dentistry. Use of polycarboxylate cements declined with the advent of glass ionomer and resin cements. The most commonly utilized class of dental cements are resin and resin-reinforced glass ionomer cements.

Resin-reinforced glass ionomer and resin cements are used in numerous dental applications, e.g., to adhere crowns, artificial implants (grouting substance), bonded bridge luting cement, for core build up, and to lute bridge work, inlays and onlays, and other related dental fixtures to the desired base, usually a mature tooth. The advent of resin cements resolved many of the retention problems experienced with the zinc-based products, presently used by practitioners. Many of the resin cements are based on acrylic polymers such as polymethyl methacrylate. Resin cements are based on acrylic or diacrylate resins and they have been used to cement crowns, conventional bridges, and resin bonded bridges; for bonding of esthetic restorations to teeth; and for direct bonding of orthodontic brackets to acid-etched enamel. The early resin cements were primarily poly(methylmethacrylate) powder with various inorganic fillers and methyl methacrylate liquid. Setting was caused by a peroxide initiator-amine accelerator system.

The self-cured, composite cements are typically powder-liquid or two paste systems. One major component is diacrylate oligomer diluted with lower molecular weight dimethacrylate monomers. The other major component is silanated silica or glass. The initiator-accelerator system is peroxide amine.

The adhesive resin cements are self-cured powder-liquid systems formulated with methacryoxyethylphenyl phosphate or 4-methacryloxyethyl-trimelitic anhydride (4-META). Phosphate cement, a two paste system, contains BIS-GMA resin and silanated quartz filler. The phosphonate is very sensitive to oxygen, so a gel is used to coat the margins of a restoration until setting has occurred. The phosphate end of the phosphonate reacts with calcium of the tooth or with a metal oxide. The 4-META cement is formulated with methyl methacrylate monomer and acrylic resin filler and is catalyzed by tri-butyl-borane. The adhesive resin cements and composite resin cements in conjunction with dentin bonding agents are being used as cements for posts and cores and veneers (porcelain and acrylic).

Many of the problems associated with resin cements is due to the acrylic resins themselves. The acrylics, particularly polymethyl methacrylate, exhibit shrinkages when polymerizing. PMMA products are extremely versatile and are utilized in a variety of dental applications.

A particularly troublesome side effect associated with the use of resin cements is post-application sensitivity. Sensitization with resin cements is attributed to swelling of the cement that occurs over time, often causing the coronal aspects of the tooth and porcelain crown to break and detach. To avoid sensitization with resin cements, it is recommended that the dentin of the tooth be hybridized prior to application of the resin cement. Hybridization is a process wherein binding or desensitizing agents are applied to the dentin of the tooth prior to cementation. Without hybridization, the potential for tooth sensitization increases.

The addition of glass ionomers to resin cements greatly improves the properties and overcomes many of the difficulties associated with previous resin cement formulations. These resin reinforced glass ionomer (RRGI) cements are, therefore, a particularly preferred class of resin cements and are the product of choice for many dental applications. RRGI cements provide excellent adhesive properties, diminishes swelling and are anti-cariogenic. The anti-cariogenic properties of certain RRGI cements are attributed to fluoride which is inherent in its composition which becomes bioavailable over time. Glass ionomer cements are supplied as a powder that is mixed with water. The liquid typically is a 47.5% solution of 2:1 polyacrylic acid/itaconic acid copolymer (average molecular weight 10,000) in water. The itaconic acid reduces the viscosity of the liquid and inhibits gelation caused by intermolecular hydrogen bonding; D(+) tartaric acid (5%, the optically active isomer) in the liquid serves as an accelerator by facilitating the extraction of ions from the glass powder. In some products, the polyacrylic acid is formulated in the powder. The liquids may be water or a dilute solution of tartaric acid in water. The setting reaction is an acid-base reaction between the acidic prolyectric and the alumino-silicate glass. The polyacid attacks the glass to release cations and fluoride ions.

The glass ionomer cements bond chemically to enamel and dentin during the setting process. The bonding mechanism is thought to involve an ionic interaction with calcium and/or phosphate ions from the surface of enamel or dentin. Treatment of dentin with a dilute solution of ferric chloride preceded by an acidic cleanser improves bonding. The cleaning agent removes the smeared layer of dentin which the Fe³+ ions are deposited and increase the ionic interaction between cement and dentin.

The powder of a glass ionomer cement is a calcium fluoraluminosilicate glass (SiO₂—AL₂O₃—CAF—NaAlF₆—ALPO₄). Known resin cement and RRGI cement formulations are described, e.g., in U.S. Pat. Nos. 4,360,605; 4,376,835, and 5,681,872.

Unfortunately, RRGI cements, like non-glass ionomer containing resin cements, have shown significant and oftentimes severe and persistent post-application sensitivity. Post-application sensitization attributed to the RRGI cements is a bothersome and oftentimes serious side-effect, and can result in removal of prostheses, root canal therapy and, in extreme cases, extraction of the affected tooth. The post-cementation sensitivity associated with RRGI cements is severe and capricious, occurring in approximately 5 to 10% of patients in which the RRGI cement is used.

To avoid this sensitization, dental clinicians prepare fast and relatively thick RRGI cement mixes. It is also known that moisturizing the teeth prior to application provides added benefit, but does not eliminate the problem.

It is known in other aspects of the dental art to incorporate a desensitizing agent into dental composition to treat hypersensitive teeth. For example, U.S. Pat. No. 5,718,885 to Gingold et al. disclose a composition for treating hypersensitive teeth containing a desensitizing agent, comprising a cationically charged colloid, e.g., CeO₂, wherein the composition is phosphate free.

U.S. Pat. No. 4,978,391 to Jones describes a method for cushioning dental appliances in the mouth using a visible light-cured polytechnic material which can also be used for intraoral delivery of a medicament. The medicament may be a non-fluoride tooth desensitizing agent such as potassium nitrate.

U.S. Pat. Nos. 4,343,608 and 4,407,675 to Hodosh describe zinc polyacrylcate cements containing potassium nitrate that are said to be healthful and useful for treating pulpitis. In one described method for preparing the cements, a freeze dried zinc polyacrylate cement powder is added to zinc oxide powder, and a small amount of a saturated aqueous solution of potassium nitrate is added. The resultant cement is applied to the desired site, where it hardens to a cement-like consistency. Glass ionomer may be added to this cement. It is noted that in some cases, a transient period of cold sensitivity existed after application, which was reported to routinely disappear wither by itself or by application of potassium nitrate paste as described in U.S. Pat. No. 3,086,006.

The present invention which is described herein overcomes the problem presented by prior art cements and the use of such prior art cements.

### OBJECTS OF THE INVENTION

It is an object of the present invention to provide dental adhesive compositions that preferably decrease the incidence and severity of sensitization compared to prior art formulations.

It is also an object of the present invention to provide methods of preparing the dental adhesive compositions of the invention.

It is further an object of the present invention to provide a method of reducing and preferably eliminating the incidence of tooth sensitization seen with prior are resin cements or RRGI cements by applying the dental adhesive composition of the present invention to the desired site.

### SUMMARY OF THE INVENTION

These objects and others are achieved by the present invention, which is related in part to dental adhesive compositions of resin dental cements and a tooth desensitizing agent. Preferably, the resin dental cement contains a glass ionomer (RRGI cement), and the tooth desensitizing agent is a potassium-containing desensitizing agent such as KNO₃.

The dental adhesive compositions of the present invention which typically include from about 1 to about 20% weight of the dental adhesive composition. However, the amount of desensitizing agent included in the composition will vary with the type of cement used and with the particular desensitizing agent. The amount of desensitizing agent will be effective to reduce the sensitization or incidence of sensitization of teeth compared to the corresponding formula without the desensitizing agent.

The present invention also provides a method of preparing the dental adhesive formulations by incorporating an amount of desensitizing agent sufficient to prevent the sensitization of teeth into a resin cement. Preferably, the desensitizing agent is added in the form of a solution, e.g., potassium nitrate solution. Incorporation of the ingredients can be effected in any manner known in the art, e.g., hand mixing, use of industrial mixing equipment, and the like.

Another embodiment of the present invention provides a method for preventing the painful sensitization of teeth by applying a dental adhesive formulation including a resin cement such as a RRGI cement and an effective amount of a desensitizing agent to the desired site of adhesion. Application of the dental adhesive compositions of the present invention reduces or prevents tooth sensitization compared to dental cement formulation lacking the desensitizing agent.

The invention is described in further detail below.

### DETAILED DESCRIPTION OF THE INVENTION

The dental adhesive compositions of the present invention include a resin cement and a sufficient amount of a desensitizing agent to reduce or prevent sensitization of the teeth following the application of resin and/or resin-reinforced glass ionomer cement to the tooth. In other preferred embodiments, the resin cement does not contain a glass ionomer. The resin found in the resin and RRGI cements preferably includes an acrylic polymer, but may include any biocompatible resin or other adhesive materials known in the art. Preferably, the resin or RRGI cement includes a polymeric resin such as polymethyl methacrylate or dimethacrylate.

It is preferred that an RRGI cement is used in the formulations of the invention, as they provide many advantages which are more fully described hereinabove. The glass ionomer used to prepare the RRGI resin may be any known to those skilled in the art, and may include various ceramic, glass-ceramic and glass ionomers or particulate substances.

The particular resin or RRGI cement used in accordance with the present invention is not critical, since it is the addition of the desensitizing agents to these resins that are the primary thrust of the present invention.

Any desensitizing agent known in the art may be used in accordance with the present invention. It is preferred that the desensitizing agent contains potassium. Suitable potassium-containing desensitizing agents include those described in U.S. Pat. No. 5,522,726 to Hodosh. A non-limiting list of preferred potassium-containing desensitizing agents includes potassium nitrate, potassium bicarbonate, potassium bromide, potassium phosphate, potassium alum, potassium sulfate, potassium chlorate, potassium fluoride, and mixtures thereof. Potassium nitrate and potassium fluoride are preferred, and potassium nitrate is particularly preferred.

Potassium nitrate is known for use as a local anesthetic in dental applications. Many different potassium nitrate compositions are known in the art, and are described, for example, in U.S. Pat. Nos. 4,407,675; 4,343,675; 4,400,373; 5,153,006; all to Hodosh.

Potassium fluoride is also a preferred desensitizing agent because fluoride is well known for its beneficial anti-caries effect. It will become physiologically available as it leaches through the dental adhesive composition into the dentinal tubules and dentin. Thus, dental adhesive formulations containing potassium fluoride are especially advantageous.

The amount of desensitizing agent included in the dental adhesive compositions of the invention will vary, but will typically be added to amounts of about 1 to about 20 wt. % of the final composition, preferably from about 1 to about 15 wt. %, and more preferably from about 1 to about 10 wt. %. It is understood, however, that the amount of desensitizing agent contained in the dental adhesive compositions of the invention will vary with the type of resin cement or RRGI cement used, with the desensitizing agent, and with other factors that will be readily apparent to those skilled in the art.

The amount of desensitizing agent incorporated in the dental adhesive product will be sufficient to decrease or prevent tooth sensitization. Of course, the amount of desensitizing agent must not detract significantly from the adhesive properties and favorable characteristics of the formulation so as to render the final product unfit or less fit for its intended purpose.

Certain formulations may require the addition of other ingredients to impart commercially desirable properties to these products, e.g., preservatives, colorants, and the like. It is preferred to include calcium, phosphate and fluoride containing compounds as these agents provide well-known beneficial effects.

The compositions of the present invention may be prepared by adding a desensitizing agent to the resin or RRGI cement. The manner of addition is not critical and may be accomplished using any technique known to the skilled artisan. Many practitioners will find it useful to prepare the dental adhesive compositions on an as-needed basis. The desensitizing agent may be incorporated as a solid, e.g., as a salt, into the powder or into the liquid component of the resin or RRGI cements.

It is preferred that if the cement is prepared as a melt, that the desensitizing agent be added in solid form. It may also be preferred to incorporate the desensitizing agent into the glass ionomer prior to the addition of the resin materials.

The dental adhesive formulations of the invention have adhesive properties suitable for use in a wide variety of dental application, and can be used in any application where a dental adhesive or dental cement is required. These cement may be used to lute crowns and bridges, as bases (interim) in permanent or semi-permanent applications to adhere dental prostheses or corrective devices to the desired site, usually an existing tooth. The skilled artisan will apply these formulations according to techniques known in the art.

The dental adhesive formulations of the present invention result in a reduced incidence and severity of sensitivity compared to formulations of the prior art. Preferably, the formulations of the present invention provide reduced incidence and severity of sensitivity compared to counterpart formulations lacking the desensitizing agent. It is preferred that the formulations of the present invention prevent sensitivity from occurring in most instances. Thus, an aspect of the present invention provides a method for preventing and reducing the incidence and severity of post-application tooth sensitivity in patients by applying the formulations of the present invention into the desired site.

Preferred embodiments of the invention are described in detail hereinbelow.

### DETAILED DESCRIPTION OF PREFERRED EMBODIMENTS

### EXAMPLE 1

100 grams of a 80% polymethyl methacrylate resin cement is prepared using art-known techniques. 50 grams of resin cement is set aside for a comparative test.

A sufficient amount of potassium nitrate is added to the remaining 50 grams of the resin to yield a dental adhesive composition containing 10 wt. % potassium nitrate. This product is tested and found to have suitable adhesive properties.

Both resins may be applied using art-known techniques to a patient in need of bilateral dental cement usage. With post-cement usage, the patient should report significant sensitivity at the site where the desensitizing-free resin cement was applied, but it is expected that the patient will not report significant sensitization on the side where the cement containing the desensitizing agent was applied.

### EXAMPLE 2

In the procedure of Example 1 is repeated in another patient in need of bilateral bridgework, except that a commercially available RRGI cement is used, the patient will likely report a slight sensitizing in the area where the non-desensitizing cement was used, but no sensitivity in the other side of treatment.

### EXAMPLE 3

A paste-paste restorative is prepared according to Example 3 of U.S. Pat. No. 5,681,872. A portion of that product was removed and potassium nitrate was added to yield a final product containing 8 wt. % potassium nitrate.

### EXAMPLE 4

An amount of a resin cement or resin-reinforced glass ionomer cement is mixed with substantially an equal amount of glass ionomer cement containing a potassium-containing desensitizing agent, such as potassium nitrate, to yield a resin cement or resin-reinforced cement with an amount of potassium nitrate. Note though that the proportion of the mixture of the resin cement or resin-reinforced glass ionomer cement and glass ionomer cement containing potassium nitrate may vary and will depend on, e.g., the concentration of potassium nitrate in the glass ionomer cement.

An amount of resin cement or resin-reinforced cement is set aside for a comparative test.

Both cements are applied using art-known techniques to a patient in need of bilateral dental cement usage. With post-cement usage, the patient reported significant sensitivity at the site where the desensitizing-free resin cement was applied, but the patient did not report significant sensitization on the side where the cement containing the desensitizing agent was applied.

Other embodiments of the invention will be readily apparent to those skilled in the art, and are contemplated to be within the scope of the present invention.

It is claimed:

1. A method for performing a restorative dental process adapted to prevent post-restorative pain comprising the steps of:
preparing a site in the oral cavity of a patient;
applying an effective amount of dental cement having from about to 1 to about 20 wt. % of a potassium-containing desensitizing agent; and
completing the dental restoration process.

2. The method of claim 1, wherein the dental cement further comprises a glass ionomer.

3. The method of claim 1, wherein said desensitizing agent is selected from the group consisting of potassium nitrate, potassium bicarbonate, potassium bromide, potassium phosphate, potassium alum, potassium sulfate, potassium chlorate, potassium chloride, potassium fluoride, and other potassium-containing compounds.

4. The method of claim 1, wherein said resin cement comprises an acrylic polymer.

5. The method of claim 4, wherein said acrylic polymer is polymethyl methacrylate.

6. The method of claim 4, wherein said acrylic powder is dimethacrylate.

7. The method of claim 4, wherein said acrylic powder is 4-META.

8. The method of claim 4, wherein said acrylic powder is BIS-MGA.

9. The method of claim 3, wherein the desensitizing agent is potassium nitrate.

10. The method of claim 9, wherein said dental cement comprises from about 1 to about 10% potassium nitrate.

11. The method of claim 1, wherein said dental cement further comprising a calcium containing compound.

12. The method of claim 1, wherein said dental cement further comprises a fluoride containing compound.

13. The method of claim 1, wherein said dental cement further comprises phosphates.

14. The method claim 1, wherein the dental cement does not contain zinc.
